(19) United States
(12) Patent Application Publication
(10) Pub. No.: US 2025/0325227 A1
(43) Pub. Date: Oct. 23, 2025

(54) APPARATUS AND METHOD FOR DIAGNOSIS AND PRESCRIPTION BASED ON ORIENTAL DIAGNOSIS PROTOCOL

(71) Applicant: KEYherblab Co., Ltd., Gangwon-do (KR)

(72) Inventor: Euy Joon Roh, Gyeonggi-do (KR)

(21) Appl. No.: 18/912,815

(22) Filed: Oct. 11, 2024

(30) Foreign Application Priority Data
Apr. 18, 2024 (KR) 10-2024-0052383

(51) Int. Cl. A61B 5/00; G16H 20/90

(52) U.S. Cl. CPC A61B 5/4854; G16H 20/90

(57) ABSTRACT

Disclosed is an oriental medicine diagnosis and prescription apparatus comprising: an identification unit that receives patient login information and identifies the patient based on the patient's login information; a first medical inquiry execution unit that makes requests to the user terminal on information about the disease or condition; an inquiry selection unit that selects one of multiple inquiry contents matching multiple disease protocols; a second medical inquiry execution unit that transmits the selected inquiry content to the user terminal; a prescription candidate group derivation unit that extracts oriental medicine herbs considering the disease protocol matching the patient's response, and derives a prescription candidate group of at least one oriental medicine preparation; and a prescription provision unit that lists the prescription candidate group and provides it to the user terminal or the corresponding Korean medicine doctor's terminal.

### CROSS-REFERENCE TO RELATED APPLICATION

This application claims priority of Korean Patent Application No. 10-2024-0052383, filed on Apr. 18, 2024, in the KIPO (Korean Intellectual Property Office), the disclosure of which is incorporated herein entirely by reference.

### BACKGROUND OF THE INVENTION

### Field of the Invention

The present invention relates to technology for diagnosing and prescribing diseases and conditions, and more specifically, to an apparatus and method for diagnosing and prescribing diseases and conditions based on oriental medicine diagnostic protocols.

### Description of the Related Art

Korean medicine doctors comprehensively understand diseases and make oriental medical diagnoses by analyzing information collected through the four diagnostic methods: visual inspection, auscultation, inquiry, and palpation. Based on these diagnoses, Korean medicine doctors treat patients through traditional Korean medicine practices passed down from ancestors, such as acupuncture, pharmacopuncture, moxibustion, cupping therapy, chuna therapy, herbal medicine, and oriental physical therapy, as well as modern applications and developments based on these traditional practices.

Recently, due to Korean medicine doctors' lack of specialized knowledge and clinical experience with herbal medicines, treatments have become primarily focused on acupuncture, resulting in a gradual decrease in herbal medicine prescriptions. Even when herbal medicines are prescribed, treatments tend to be based on simple symptom-based prescriptions. These prescriptions are based on traditional herbal medicine prescriptions, but since the historical prescriptions themselves are written in difficult Chinese characters, it is challenging to apply these historical prescriptions in clinical practice, and this lack of clinical experience may result in reduced treatment effectiveness.

Meanwhile, the invention disclosed in prior patent document KR 10-2044079 reveals a method and apparatus for searching oriental medicine prescriptions. The candidate prescriptions disclosed in the prior patent document extract matching candidate prescriptions by having users manually input search conditions including herbs to be included, number of herbs, formulation types, and source references. The candidate prescriptions disclosed in this prior patent document merely provide a user interface and database for searching oriental medicine prescriptions regardless of the patient's symptoms or condition, and there is a need for an improved method that may automatically generate candidate prescriptions according to the patient's symptoms.

Furthermore, there is a need to provide standardized and quantified prescription criteria to Korean medicine doctors so that they may prescribe highly effective herbal medicines even if they lack specialized knowledge of medicinal herbs.

The above-described information disclosed in the background description is provided only for improving the understanding of the background of the present disclosure, and thus may include the information which does not constitute the prior art.

### SUMMARY OF THE INVENTION

The technical problem to be solved by the present invention is to provide a personalized oriental medicine diagnosis and prescription apparatus and method that automatically provides candidate prescriptions according to each patient's symptoms.

Additionally, it is to provide an oriental medicine diagnostic protocols-based apparatus and method for diagnosis and prescription that provides standardized and quantified prescription criteria to both patients and Korean medicine doctors.

The problems to be solved by the present invention are not limited to the problems mentioned above, and other problems not mentioned will be understood by those skilled in the art from the following description.

According to an embodiment of the present invention, an oriental medicine diagnosis and prescription apparatus may be provided, comprising: an identification unit that receives patient login information from a user terminal and identifies the patient based on the patient's login information; a first medical inquiry execution unit that makes requests to the user terminal on information about the disease or condition that the identified patient wants to be treated for; an inquiry selection unit that selects one of multiple inquiry contents matching multiple disease protocols according to the patient's response to the request; a second medical inquiry execution unit that transmits the selected inquiry content to the user terminal step by step according to a predetermined order; a prescription candidate group derivation unit that extracts oriental medicine herbs considering the disease protocol matching the patient's response to the request and the patient's response to the selected inquiry content, and derives a prescription candidate group of at least one oriental medicine preparation corresponding to the extracted herbs; and a prescription provision unit that lists the prescription candidate group and provides it to the user terminal or the corresponding Korean medicine doctor's terminal.

The disease protocols may include at least one of: insomnia protocol, psychiatric protocol, allergic or non-allergic rhinitis protocol, cold protocol, shortness of breath or asthma protocol and chronic cough protocol, facial nerve palsy protocol, cerebrovascular disease sequelae protocol, menstrual pain protocol, gynecological protocol, lumbar disc herniation (back disc) protocol, other musculoskeletal pain protocol, or functional dyspepsia protocol, other digestive system protocol, wherein the psychiatric protocol includes any one of panic disorder, anxiety disorder, obsessive-compulsive disorder, depression, schizophrenia, tic disorder, and epilepsy, and the gynecological protocol includes any one of infertility, amenorrhea, and menorrhagia, and the other musculoskeletal pain protocol includes any one of cervical disc, spinal stenosis, arthritis, and frozen shoulder, and the other digestive system protocol may include any one of constipation, diarrhea, and gastritis.

The insomnia protocol may prescribe based on the selected inquiry content by: categorizing the patient into triggering type and aggravating type, and determining guiding herbs and weak emotions according to the patient's duration and frequency of illness, wherein:
When it is triggering type with long duration, severe symptoms, and frequent occurrence, ‘Coptis chinensis’ is used as the guiding herb and the weak emotion state is excitement and anger;
When it is triggering type with short duration, mild symptoms, and occasional occurrence, at least one of ‘Ostrea Gigas’ and ‘Fossilia Ossis Mastodi’ is used as the guiding herb and the weak emotion state is anxiety or fear;
When it is aggravating type with long duration, severe symptoms, and frequent occurrence, ‘Fermented Mass’ or ‘Gardenia jasminoides’ is used as the guiding herb and the weak emotion state is resentment and sadness;
When it is aggravating type with short duration, mild symptoms, and occasional occurrence, ‘Poria cocos’ or ‘Zizyphus jujuba’ is used as the guiding herb and the weak emotion state is worry.

‘Coptis chinensis’ and ‘Ostrea Gigas’ are prescribed for patients with Yin constitution, while ‘Fermented Mass’, ‘Gardenia jasminoides’, ‘Poria cocos’, ‘Zizyphus jujuba’, and ‘Fossilia Ossis Mastodi’ may be prescribed for patients with Yang constitution.

The rhinitis protocol may prescribe based on the selected inquiry content by: categorizing the patient into watery, mucous, purulent, and dry types, and determining guiding herbs and their associated paired herbs according to aggravating and alleviating factors, wherein: for watery type with cold stimulus and aggravation triggers, at least one of ‘Ephedra Sinica’ and ‘Asarum sieboldii’ is used as the main guiding herb, and ‘Zingiber Officinale’ is used as the secondary guiding herb. For watery type with heat stimulus and relief loss, at least one of ‘Ephedra Sinica’ and ‘Asarum Sieboldii’ is used as the main guiding herb, and ‘Leonurus Japonicus’ is used as the secondary guiding herb; For mucous type with seasonal characteristics, ‘Pinellia ternata’ is used as the main guiding herb, and ‘Gleditsia sinensis’ is used as the secondary guiding herb. For purulent type with year-round occurrence regardless of cold or heat stimulus, ‘Platycodon Grandiflorum’ is used as the main guiding herb, and ‘Coix lacryma-jobi’ is used as the secondary guiding herb. For dry type triggered and aggravated by dry environment and relieved by humid environment, ‘Scutellaria Baicalensis’ is used as the main guiding herb, and ‘Ophiopogon japonicus’, ‘Schisandra chinensis’, and ‘Prunus Armeniaca’ are used as the secondary guiding herbs. For watery rhinitis, at least one of ‘Pinellia ternata’, ‘Schisandra chinensis’, and ‘Zingiber Officinale’ is used as paired herbs, and when nasal congestion symptoms are present in watery rhinitis, at least one of ‘Magnolia liliiflora’, ‘Ligusticum chuanxiong’, ‘Xanthium strumarium’, and ‘Angelica dahurica’ is used as paired herbs, and for purulent rhinitis, at least one of ‘Gypsum Fibrosum’, ‘Scutellaria baicalensis’, ‘Pueraria lobata’, and ‘Belamcanda chinensis’ may be used as heat-clearing paired herbs.

The shortness of breath or asthma protocol prescribes based on the selected inquiry content by: categorizing the patient into watery, purulent, mucous, and dry types, and determining guiding herbs according to aggravating and alleviating factors, wherein: for watery type where Ephedra may be used, ‘Ephedra sinica’ is used as the main guiding herb; For watery type where Ephedra may not be used, ‘Cnidium officinale’ is used as the main guiding herb, and ‘Zizyphus jujuba’ is used as the secondary guiding herb; For purulent type not significantly affected by cold or heat stimulus, ‘Platycodon grandiflorum’ is used as the guiding herb; For mucous type with chest discomfort, at least one of ‘Trichosanthes kirilowii’, ‘Magnolia officinalis’, and ‘Prunus armeniaca’ is used as the main guiding herb, and ‘Pinellia ternata’ or ‘Prunus armeniaca’ is used as the secondary guiding herb. For dry type with chest discomfort and thoracic fullness, ‘Citrus unshiu Peel’ is used as the guiding herb, and for dry type with leg bone damage and lumbar edema, ‘Sinomenium acutum’ is used as the guiding herb. When using ‘Ephedra sinica’ as the guiding herb for watery shortness of breath or asthma, one of Munhap-tang (Korean herbal decoction combining harmonizing herbs), Gamcho-mawhang-tang (Licorice-Ephedra Decoction), Mawhang-buja-gamcho-tang (Ephedra, Aconite and Licorice Decoction), Sagan-mawhang-tang (Belamcanda and Ephedra Decoction), Bangki-hwangki-gamawhang-tang (Sinomenium, Astragalus and Ephedra Decoction), Oedaesinbi-tang (Major Decoction for Body Revitalization) is prescribed; When using ‘Cnidium officinale’ as the guiding herb for watery shortness of breath or asthma, one of Jeongryeok-daejo-sapye-tang (Descurainia and Jujube to Drain the Lungs Decoction), Gichoryeokhwanghwan Pills (Basic Cnidium and Rhubarb Pills), or Daeham-heung-hwan (Major Chest-Relieving Pills) is prescribed. When using ‘Pinellia ternata’, ‘Trichosanthes kirilowii’, ‘Magnolia officinalis’, or ‘Perilla frutescens’ as the guiding herb for mucous shortness of breath or asthma, one of Soham-heung-tang (Minor Chest Relief Decoction), Gwaru-haebaek-banha-tang (Trichosanthes, Fritillaria and Pinellia Decoction), Banha-hubak-tang (Pinellia and Magnolia Bark Decoction), or Soja-dodam-ganggi-tang (Minor Phlegm-Resolving and Qi-Strengthening Decoction) is prescribed; When using ‘Prunus armeniaca’, ‘Citrus unshiu Peel’, or ‘Sinomenium acutum’ as the guiding herb for dry shortness of breath or asthma, one of Gyeji-gahubak-haengja-tang (Cinnamon Twig, Magnolia Bark and Apricot Kernel Decoction), Bokryeong-haengin-gamcho-tang (Poria, Apricot Kernel and Licorice Decoction), Gyulpi-jukyeo-tang (Citrus Peel and Bamboo Decoction), Gyulpi-jisil-saenggang-tang (Citrus Peel, Poncirus and Ginger Decoction), Mokbanggi-tang (Woody Sinomenium Decoction), Mokbanggi-geoseokgo-gayeongcho-tang (Woody Sinomenium with Gypsum and Honeysuckle Decoction), Gicho-ryeokhwang-hwan (Basic Cnidium and Rhubarb Pills), or Bangki-hwangki-gamawhang-tang (Sinomenium, Astragalus and Ephedra Decoction) may be prescribed.

The chronic cough protocol prescribes based on the selected inquiry content by: categorizing the patient into watery, mucous, purulent, and dry types, and determining guiding herbs and their associated paired herbs according to aggravating and alleviating factors. For watery type with cold stimulus and aggravation triggers, at least one of ‘Ephedra sinica’ and ‘Asarum sieboldii’ is used as the main guiding herb, and at least one of ‘Zingiber Officinale’ and ‘Cnidium Officinale’ is used as the secondary guiding herb; For mucous type with heat stimulus and relief loss, ‘Pinellia ternata’ is used as the main guiding herb, and at least one of ‘Gleditsia sinensis’, ‘Inula Flower’, and ‘Sinapis alba’ is used as the secondary guiding herb; For purulent type not significantly affected by cold or heat stimulus, ‘Platycodon grandiflorum’ is used as the main guiding herb, and at least one of ‘Trichosanthes kirilowii’, ‘Fritillaria’, ‘Angelica decursiva’, and ‘Phyllostachys nigra’ is used as the secondary guiding herb. For dry type triggered and aggravated by dry environment and relieved by humid environment, ‘Scutellaria baicalensis’ is used as the main guiding herb, and at least one of ‘Schisandra chinensis’, ‘Prunus armeniaca’, and ‘Rehmannia glutinosa’ is used as the secondary guiding herb. For the chronic cough protocol, at least one of the following may be prescribed as antitussive and expectorant paired herbs: Perilla frutescens, Perilla Seed, Purple Perilla, Tussilago farfara (Coltsfoot), Mulberry Leaf, and Mulberry Root Bark.

The musculoskeletal pain protocol prescribes based on the selected inquiry content by: categorizing the patient into primary and secondary pathogenic factors, where the primary factors are divided into cold, heat, edema, and muscle tension, and the secondary factors are divided into blood stasis, nutritional deficiency, phlegm-dampness, qi stagnation, and spirit. For primary factor with cold, ‘Cinnamomum cassia’ is used as the main guiding herb, and ‘Evodia rutaecarpa’ or ‘Aconitum carmichaelii’ is used as the secondary guiding herb; For primary factor with heat, at least one of ‘Gypsum Fibrosum’, ‘Glycyrrhiza uralensis’, ‘Scutellaria baicalensis’ is used as the main guiding herb, and one of ‘Bupleurum chinense’, ‘Coptis chinensis’ or ‘Phellodendron amurense’, ‘Gardenia jasminoides’. For primary factor with edema, at least one of ‘Tetrapanax papyriferus’, ‘Sinomenium acutum’, ‘Glycyrrhiza uralensis’ is used as the main guiding herb, and one of ‘Astragalus membranaceus’, ‘Ephedra sinica’ is used as the secondary guiding herb. For primary factor with muscle tension, ‘Ziziphus jujuba’ or ‘Paeonia lactiflora’ is used as the guiding herb. For secondary factor with blood stasis and deficiency pattern, ‘Paeonia lactiflora’ is used as the guiding herb. For secondary factor with blood stasis and excess pattern, ‘Rheum officinale’ is used as the guiding herb; For secondary factor with nutritional deficiency, ‘Rehmannia glutinosa’ is used as the guiding herb. For secondary factor with phlegm-dampness, at least one of ‘Pinellia ternata’, ‘Citrus reticulata’, ‘Arisaema amurense’ is used as the guiding herb. For secondary factor with qi stagnation, at least one of ‘Cyperus rotundus’, ‘Perilla frutescens’, ‘Citrus reticulata’, ‘Lindera aggregata’, ‘Citrus aurantium’ is used as the guiding herb. For secondary factor with spirit and yang pattern, ‘Coptis chinensis’ is used as the main guiding herb, ‘Ostrea gigas’ and Baihu Tang-type formulas are used as the secondary guiding herbs. For secondary factor with spirit and yin pattern, at least one of ‘Poria cocos’, ‘Fermented Mass’, ‘Gardenia jasminoides’ is used as the main guiding herb, and ‘Fossilia Ossis Mastodi’ is used as the secondary guiding herb. ‘Anemarrhena asphodeloides’ is used as the secondary guiding herb.

In one embodiment, the prescription candidate group derivation unit may derive a primary prescription candidate group of oriental medicine preparations corresponding to the extracted medicinal herbs, map proof of evidence to the primary prescription candidate group of the extracted oriental medicine preparations, and derive a secondary prescription candidate group of oriental medicine preparations by differentiating the primary prescription candidate group of the extracted oriental medicine preparations by grades and rankings.

Additionally, it includes an oriental medicine database that stores and maintains information about each of the multiple disease protocols and the multiple inquiry contents matching these disease protocols, and the oriental medicine database may further store prescribed herbal medicine prescriptions for each individual patient and the degree of symptom recovery feedback from the patient, to be used for artificial intelligence learning. Based on the artificial intelligence learning, the corresponding disease protocols and inquiry contents may be updated and modified.

According to an embodiment of the present invention, a method for oriental medicine diagnosis and prescription may be provided, comprising: q stage of receiving login information of a patient from a user terminal and identifying the patient based on the patient's login information; a stage of requesting information about the disease or condition that the identified patient wants to be treated for from the user terminal; a stage of selecting one of multiple inquiry contents matching multiple disease protocols according to the patient's response to the request; a stage of transmitting the selected inquiry content to the user terminal step by step according to a predetermined order; a stage of extracting medicinal herbs considering the disease protocol matching the patient's response to the request and the patient's response to the selected inquiry content, and deriving a prescription candidate group of at least one oriental medicine preparation corresponding to the extracted medicinal herbs; and a stage of listing the prescription candidate group and providing it to the user terminal or the corresponding Korean medicine doctor's terminal.

According to the embodiments of the present invention, by including a prescription candidate group derivation unit that extracts medicinal herbs considering the disease protocol matching the patient's responses and the patient's responses to inquiry contents, and derives a prescription candidate group of at least one oriental medicine preparation corresponding to the extracted herbs, the invention may provide a personalized oriental medicine diagnosis and prescription apparatus that automatically provides candidate prescriptions according to each patient's symptoms.

Furthermore, it may provide oriental medical diagnosis and prescription device that provides standardized and quantified prescription criteria to patients and oriental medicine doctors.

Furthermore, it may provide oriental medical diagnosis and prescription method that has the aforementioned advantages.

However, the effects of the present invention are not limited to the above effects, and may be diversely expanded within a scope not departing from the technical spirit and scope of the present invention.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a configuration diagram of an oriental medical diagnosis and prescription device according to an embodiment of the present invention.

FIG. 2 is a diagram illustrating an artificial intelligence model for oriental medical diagnosis and prescription.

FIG. 3 is a flowchart explaining an oriental medical diagnosis and prescription method according to an embodiment of the present invention.

FIG. 4 is a diagram showing an example of an insomnia protocol according to an embodiment of the present invention.

FIG. 5A is a diagram showing an example of a rhinitis protocol according to an embodiment of the present invention.

FIG. 5B is a diagram showing a rhinitis prescription map according to the rhinitis protocol of an embodiment of the present invention.

FIG. 5C and FIG. 5D are diagrams showing medical questionnaires according to the rhinitis protocol of an embodiment of the present invention.

FIG. 6A is a diagram showing an example of a shortness of breath protocol according to an embodiment of the present invention.

FIG. 6B is a diagram showing a shortness of breath prescription map according to the shortness of breath protocol of an embodiment of the present invention.

FIG. 7A is a diagram showing an example of a chronic cough protocol according to an embodiment of the present invention.

FIG. 7B is a diagram showing a chronic cough prescription map according to the chronic cough protocol of an embodiment of the present invention.

FIGS. 7C and 7D are diagrams showing medical questionnaire contents according to the chronic cough protocol of an embodiment of the present invention.

FIGS. 8A, 8B, 8C, and 8D are diagrams showing examples of a musculoskeletal pain disease protocol according to an embodiment of the present invention.

FIGS. 8E, 8F, and 8G are diagrams showing musculoskeletal pain disease prescriptions according to the musculoskeletal pain disease protocol of an embodiment of the present invention.

In the following description, the same or similar elements are labeled with the same or similar reference numbers.

### DETAILED DESCRIPTION

Hereinafter, a preferred embodiment of the present disclosure will be elucidated in detail with reference to the accompanying drawings.

The embodiments of the present disclosure are provided for more completely explaining the present disclosure to those skilled in the art, the below embodiments can be modified to various forms and the scope of the present disclosure is not limited to the below embodiments. These embodiments are rather provided for more faithfully and completely explaining the present disclosure and for completely conveying the spirit of the present disclosure to those skilled in the art.

In the drawings, in addition, the dimension or thickness of each layer is exaggerated for clarity and convenience of the description and the same reference numeral indicates the same structural element. As used in the detail description, the term “and/or” includes any one of the listed items and one or more combination thereof. In addition, the term “connect” in the detail description means the state in which A member is directly connected to B member as well as the state in which C member disposed between A member and B member so that A member is indirectly connected to B member via C member.

The terms used herein are employed for describing the specific embodiment and the present disclosure is not limited thereto. As used in the detailed description and the appended claims, the singular forms may include the plural forms as well, unless the context clearly indicates otherwise. In addition, the terms “comprises” and/or “comprising” or “includes” and/or “including” used in the detailed description specify the presence of stated features, regions, integers, steps, operations, elements, and/or components, but do not preclude the presence or addition of one or more other features, regions, integers, steps, operations, elements, components, and/or groups thereof.

Although the terms of “first”, “second”, etc. are used herein to describe various members, parts, regions, layers and/or sections, it is obvious that these members, parts, regions, layers and/or sections should not be limited by the above terms. These terms are employed only for distinguishing one member, part, region, layer or section from another region, layer or section. Thus, the first member, the first part, the first region, the first layer or the first section described below may refer to the second member, the second part, the second region, the second layer or section without departing from the teachings of the present disclosure.

Furthermore, the terms related to a space such as “beneath”, “below”, “lower”, “above” and “upper” may be used to easily understand one element or a characteristic or another element or a characteristic illustrated in the drawings. The above terms related to the space are employed for easy understanding of the present disclosure depending on various process states or usage states of the present disclosure, and are not intended to limit the present disclosure.

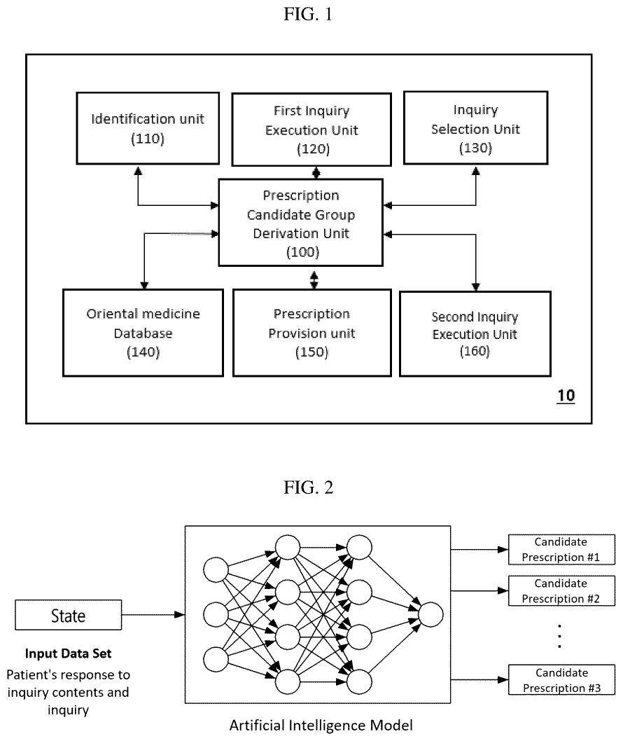

FIG. 1 is a configuration diagram of an oriental medicine diagnosis and prescription apparatus 10 according to an embodiment of the present invention.

Referring to FIG. 1, the oriental medicine diagnosis and prescription apparatus 10 may include: an identification unit 100 that receives patient login information from a user terminal and identifies the patient based on the patient login information; a first medical inquiry execution unit 120 that requests information about the disease or condition that the identified patient wants to be treated for from the user terminal; a medical inquiry selection unit 130 that selects one of multiple medical inquiry contents matching multiple disease protocols according to the patient's response to the request; a second medical inquiry execution unit 160 that transmits the selected medical inquiry content to the user terminal step by step according to a predetermined order; a prescription candidate group derivation unit 100 that extracts medicinal herbs considering the patient's response matching the disease protocol and the patient's response corresponding to the selected medical inquiry content, and derives a prescription candidate group of at least one herbal medicine preparation corresponding to the extracted medicinal herbs; and a prescription provision unit 150 that lists the prescription candidate group and provides it to the user terminal or the corresponding oriental medicine doctor terminal. Non-limitingly, the disease protocol may include at least one of insomnia protocol, psychiatric protocol, allergic or non-allergic rhinitis protocol, cold protocol, shortness of breath or asthma protocol and chronic cough protocol, facial nerve palsy protocol, post-cerebrovascular disease sequelae protocol, menstrual pain protocol, gynecology protocol, lumbar disc herniation (herniated disc) protocol, other musculoskeletal pain protocol or functional dyspepsia protocol, and other digestive system protocol.

The psychiatric protocol may include any one of panic disorder, anxiety disorder, obsessive-compulsive disorder, depression, schizophrenia, tic disorder, and epilepsy, and the gynecology protocol may include any one of infertility, amenorrhea, and metrorrhagia, and the other musculoskeletal pain protocol may include any one of cervical disc, spinal stenosis, arthritis, and frozen shoulder, and the other digestive system protocol may include any one of constipation, diarrhea, and gastritis.

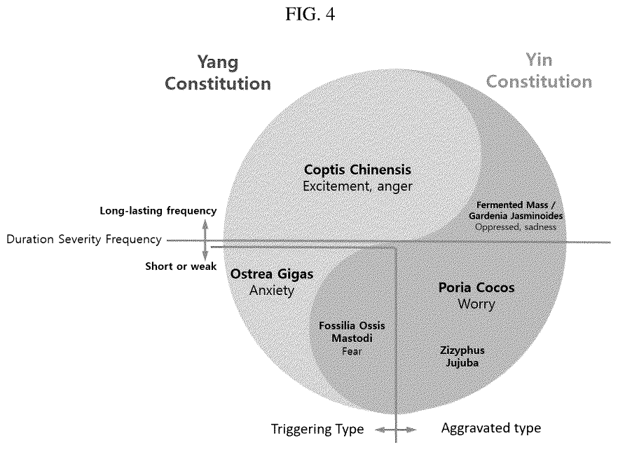

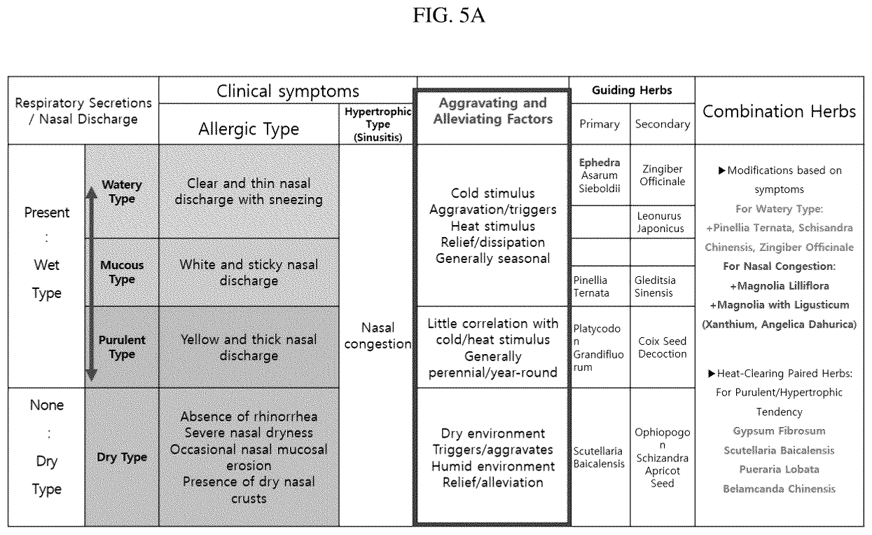

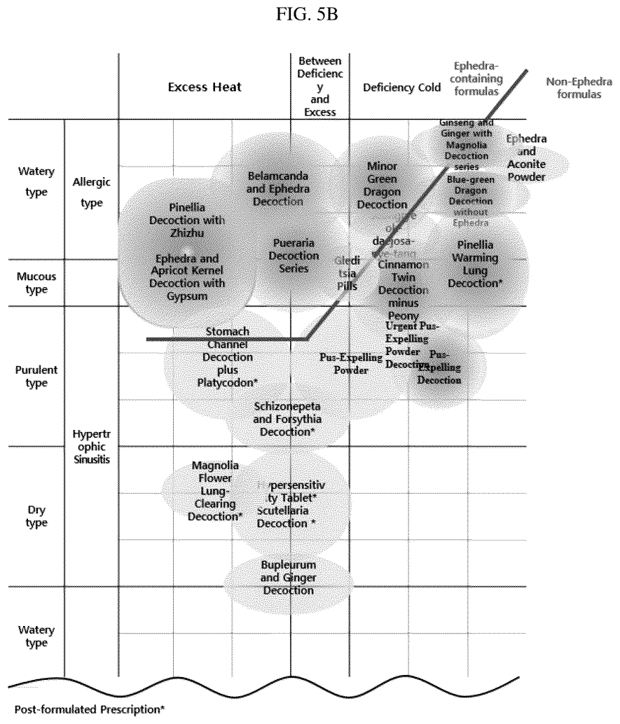

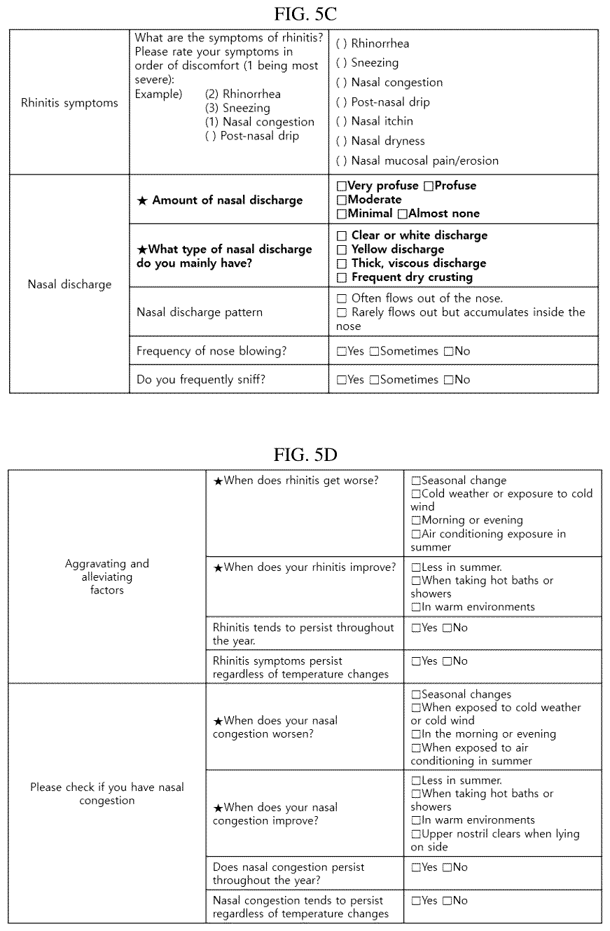

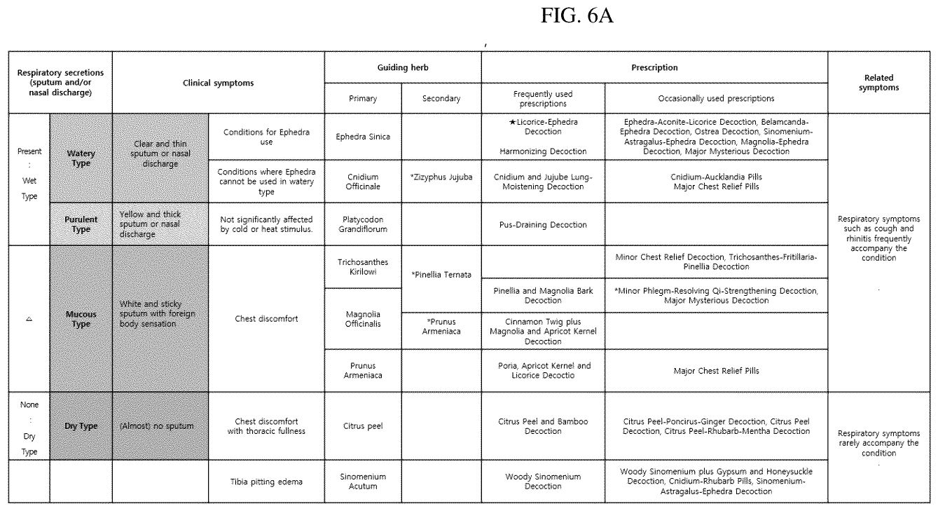

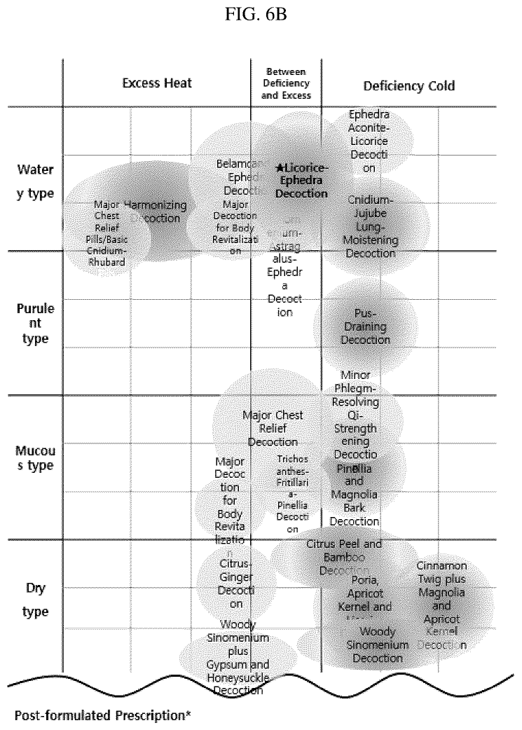

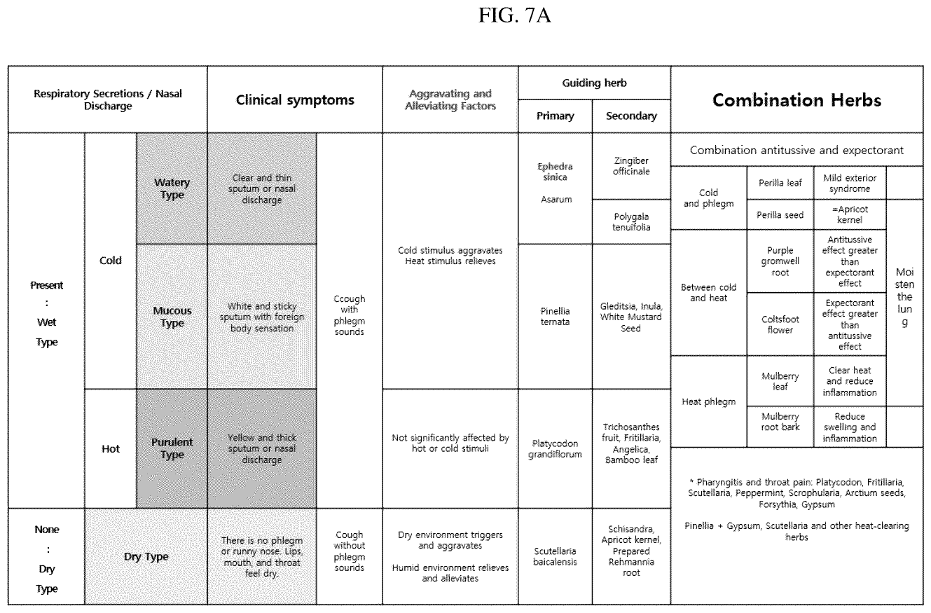

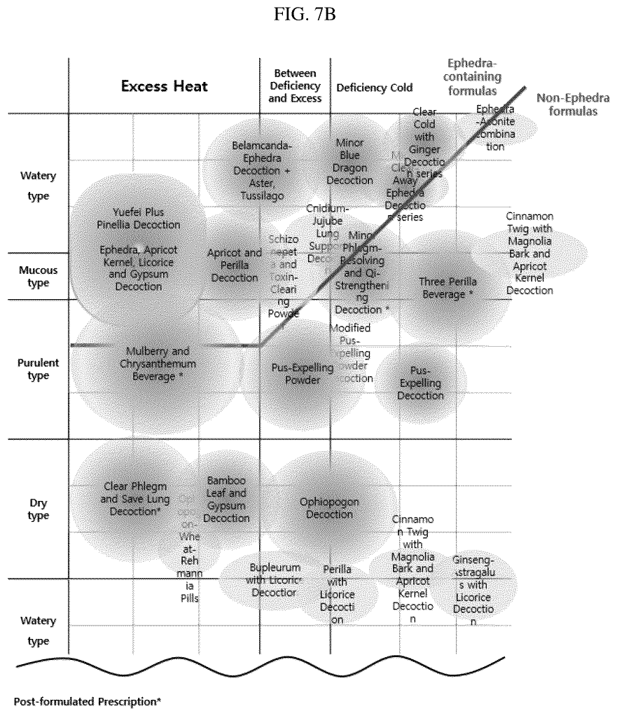

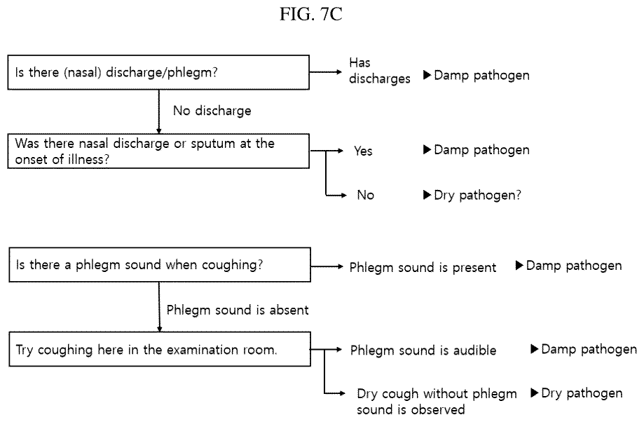

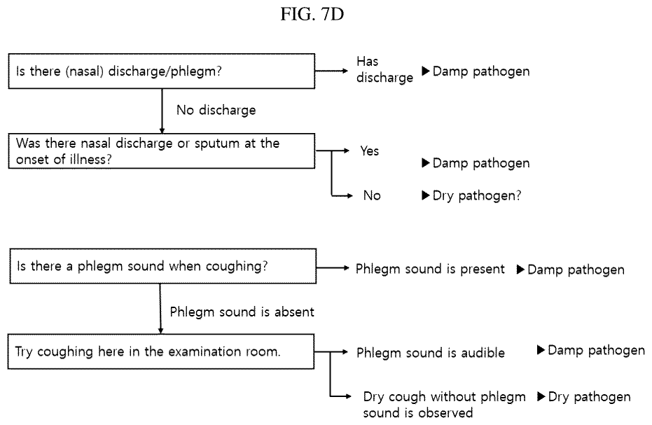

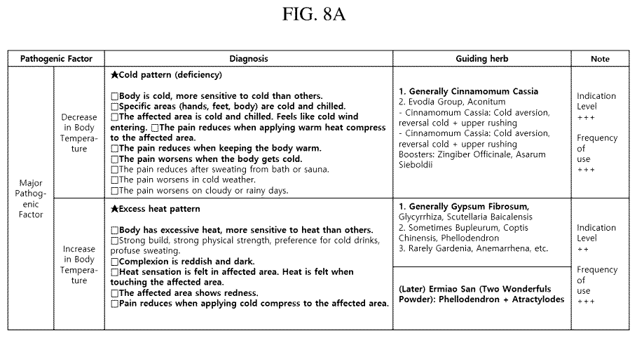

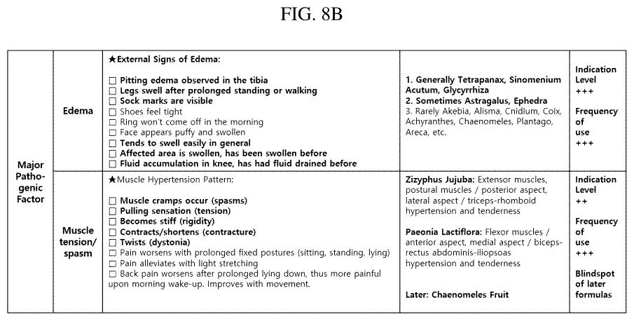

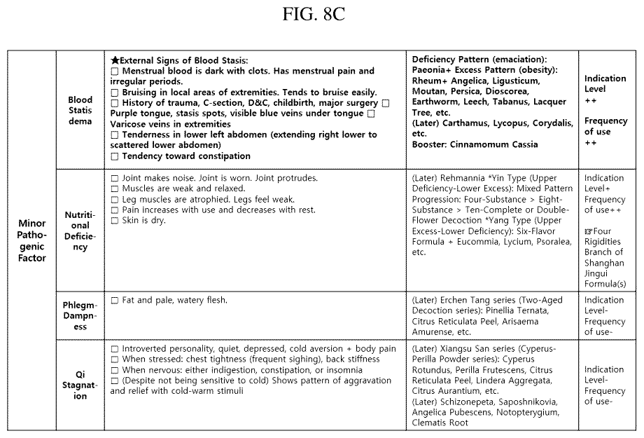

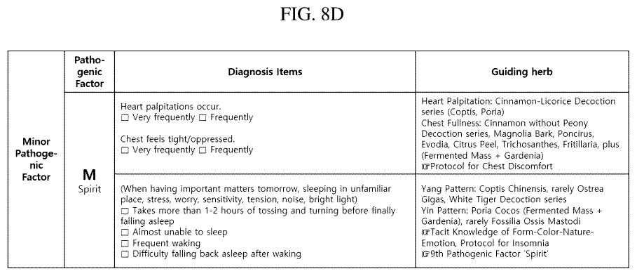

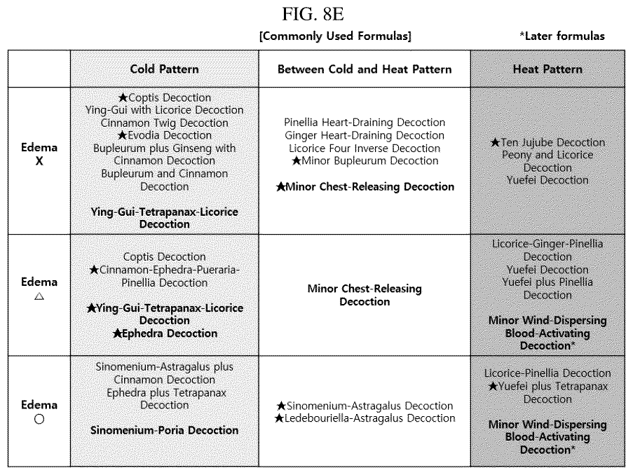

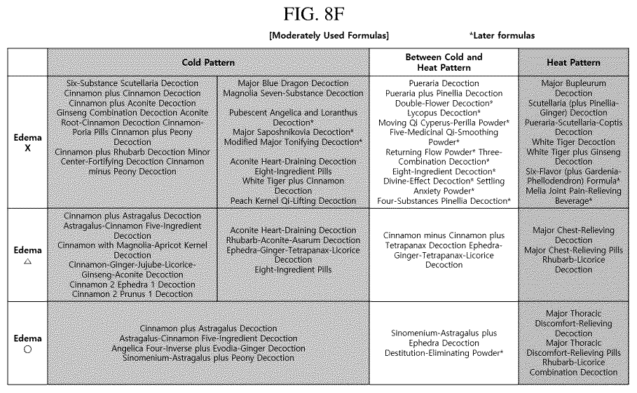

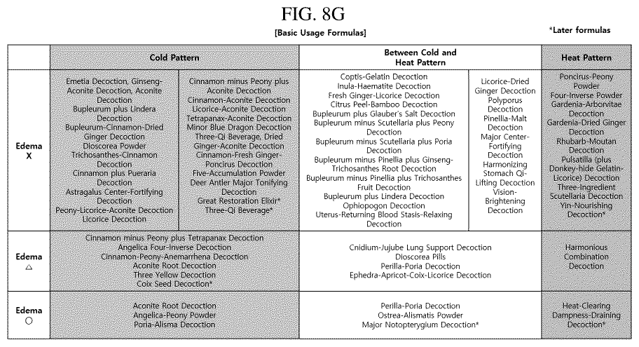

The insomnia protocol may provide candidate prescriptions for treating patient's insomnia according to the protocol expressed in FIG. 4 to be described later. Detailed description of the insomnia protocol may be referenced in FIG. 4. The rhinitis protocol may provide candidate prescriptions for treating patient's rhinitis according to the protocol expressed in FIGS. 5A-5C to be described later. Detailed description of the rhinitis protocol may be referenced in FIGS. 5A-5C. The shortness of breath or asthma protocol may provide candidate prescriptions for treating patient's shortness of breath or asthma according to the protocol expressed in FIGS. 6A and 6B to be described later. Detailed description of the shortness of breath or asthma protocol may be referenced in FIGS. 6A and 6B. The chronic cough protocol may provide candidate prescriptions for treating patient's chronic cough according to the protocol expressed in FIGS. 7A-7D to be described later. Detailed description of the chronic cough protocol may be referenced in FIGS. 7A to 7D. The musculoskeletal pain disease protocol may provide candidate prescriptions for treating patient's musculoskeletal pain disease according to the protocol expressed in FIGS. 8A-8G to be described later. Detailed description of the musculoskeletal pain disease protocol may be referenced in FIGS. 8A-8G.

In one embodiment, the prescription candidate group derivation unit 150 may derive a primary prescription candidate group of herbal medicine preparations corresponding to the extracted medicinal herbs, map proof of evidence to the primary prescription candidate group of the extracted herbal medicine preparations, and derive a secondary prescription candidate group of herbal medicine preparations by differentiating the primary prescription candidate group of the extracted herbal medicine preparations by grade and rank.

It may further include an Oriental medicine database 140 that stores and maintains information about each of the multiple disease protocols and multiple medical inquiry contents matching the multiple disease protocols. The Oriental medicine database 140 may further store herbal medicine prescriptions prescribed to each individual patient and the degree of symptom recovery fed back from the patient for use in artificial intelligence learning. Furthermore, the corresponding disease protocol and medical inquiry content may be updated and modified according to the artificial intelligence learning.

According to an embodiment of the present invention, the first medical inquiry execution unit 120 may acquire information about the disease name or condition that the patient identified by the identification unit 110 wants to be treated for and provide the result to the medical inquiry selection unit 130. The medical inquiry selection unit 130 may extract only the medical inquiry contents corresponding to the disease name or condition that the identified patient wants to be treated for from among the medical inquiry contents for multiple disease names or conditions stored in the oriental medicine database 140 and provide them to the second medical inquiry execution unit 160. The second medical inquiry execution unit 160 conducts medical inquiries step by step through questions and answers with the patient according to a predetermined order, using the medical inquiry content corresponding to the disease name or condition that the identified patient wants to be treated for, and receives answers to the medical inquiries from the patient according to the symptoms of the disease.

Non-limitingly, the order of questions to the patient may be configured to ask sequentially according to questions related to mental state, questions related to input entering the human body or output coming out from the human body, and temperature (whether sensitive to cold or heat, and temperature of hands, feet, and lower abdomen). The medical inquiry data may be standardized through standardization and quantification, and may also be modified through patient feedback as described later.

Subsequently, the prescription candidate group derivation unit 100 may derives herbal medicine data through sequential extraction of medicinal herbs (guiding herbs) related to the medical inquiry data according to the medical inquiry data (patient's answers to questions) transmitted from the second medical inquiry execution unit 160, and derives primary prescription candidate group data of herbal medicine preparations related to the derived herbal medicine data. The herbal medicine data and primary prescription candidate group data of herbal medicine preparations may be derived using the Oriental medicine database 140. The transmitted medical inquiry data is associated with any one of the insomnia protocol, rhinitis protocol, shortness of breath or asthma protocol, and chronic cough protocol to be described later, and the guiding herbs defined in the corresponding disease protocol may be determined from the patient's answers corresponding to the medical inquiry content. The Oriental medicine database 140 stores medical inquiry content according to protocols corresponding to each individual disease, and maintains information about medicinal herbs (guiding herbs) and related combination herbs according to each disease protocol.

The prescription candidate group derivation unit (100) may generate herbal medicine data by sequentially extracting medicinal herbs that should be included in and excluded from herbal medicine preparations from the Oriental medicine database 140 at each medical inquiry step using the medical inquiry data acquired according to the sequential medical inquiries of the second medical inquiry execution unit 160. Furthermore, the prescription candidate group derivation unit 100 may extract and generate herbal medicine preparation data of the primary prescription candidate group by matching and comparing herbal medicine preparations that mutually match with the extracted herbal medicine data.

The oriental medicine database 140 includes herbal medicine preparation data related to oriental medicine prescriptions, herbal medicine preparation data mutually matching with herbal medicine data, various types of contraindication data having correlation with each herbal medicine preparation, and herbal medicine prescription standard data that may present standards for herbal medicine prescriptions may be stored.

As an example, in the data table of ‘Herbal Medicine Preparation A’, there exists a data table of ‘Medicinal Herb a’ of ‘a={a1, a2, a3}’ that is mutually matched with ‘Herbal Medicine Preparation A’, and a data table of ‘5 Types of proof of evidence’ of ‘t={t1, t2, t3, t4, t5}’ that is associated with ‘Herbal Medicine Preparation A’. Therefore, by comparing various types of medicinal herb data extracted from the prescription candidate group derivation unit 100 with the oriental medicine database 140, multiple herbal medicine preparations related to the extracted medicinal herbs may be derived.

Meanwhile, the oriental medicine database 140 may generate herbal medicine preparation data mapped with proof of evidence by mapping various types of proof of evidence to the herbal medicine preparation data of the primary prescription candidate group extracted through sequential medical inquiries of the second medical inquiry execution unit 160 using the oriental medicine database 140, derive herbal medicine preparation data of the secondary prescription candidate group by differentially applying the herbal medicine preparation data mapped with proof of evidence by grade and rank, and finally generate a prescription list.

Referring to FIG. 2, multiple datasets for learning may be prepared and input first. The datasets may consist of information about protocols for treating each disease and inquiry contents in the medical questionnaire for each corresponding disease. When datasets are input into the artificial intelligence model, they may be candidate prescription groups prescribed for treating the patient's disease. The artificial intelligence model may be trained by receiving feedback about the prescriptions prescribed to patients and their condition after taking the prescriptions from the patients.

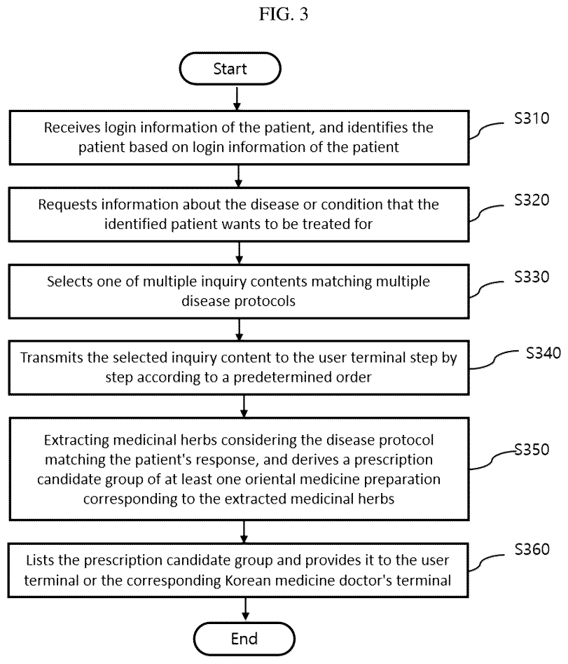

FIG. 3 is a flowchart explaining the oriental medicine diagnosis and prescription method according to an embodiment of the present invention.

Referring to FIG. 3, an Oriental medicine diagnosis and prescription method may be provided including: receiving patient login information from a user terminal and identifying the patient based on the patient login information (S310); requesting information about the disease name or condition that the identified patient wants to be treated for from the user terminal (S320); selecting one of multiple medical inquiry contents matching multiple disease protocols according to the patient's response to the request (S330); transmitting the selected medical inquiry content to the user terminal step by step according to a predetermined order (S340); extracting medicinal herbs considering the patient's response matching the disease protocol and the patient's response corresponding to the selected medical inquiry content, and deriving a prescription candidate group of at least one herbal medicine preparation corresponding to the extracted medicinal herbs (S350); and listing the prescription candidate group and providing it to the user terminal or the corresponding Oriental medicine doctor terminal (S360).

FIG. 4 is a diagram showing an example of an insomnia protocol according to an embodiment of the present invention.

Referring to FIG. 4, based on the patient's feedback information about medical inquiry content associated with the insomnia protocol, the insomnia protocol may classify the patient into inducing type and aggravating type, and determine guiding herbs and weak emotion according to the patient's duration and frequency of illness.

Specifically, in cases where the patient is of inducing type and shows long duration, severe symptoms, and frequent occurrence, ‘Coptis rhizome’ is used as the guiding herb and the weak emotion state is excitement and anger. In cases where the patient is of inducing type and shows short duration, mild symptoms, and occasional occurrence, at least one of ‘Ostrea Gigas’ and ‘Fossilia Ossis Mastodi’ is used as the guiding herb and the weak emotion state is anxiety or fear. In cases where the patient is of aggravating type and shows long duration, severe symptoms, and frequent occurrence, ‘Xiang Shi’ or ‘Gardenia’ is used as the guiding herb and the weak emotion state is resentment and sadness. In cases where the patient is of aggravating type and shows short duration, mild symptoms, and occasional occurrence, ‘Poria cocos’ or ‘Zizyphus spinosa’ is used as the guiding herb and the weak emotion state is worry. In this case, ‘Coptis rhizome’ and ‘Ostrea Gigas’ are prescribed to patients with Yin constitution, and ‘Xiang Shi’, ‘Gardenia’, ‘Poria cocos’, ‘Zizyphus spinosa’, and ‘Fossilia Ossis Mastodi’ may be prescribed to patients with Yang constitution.

FIG. 5A is a diagram showing an example of a rhinitis protocol according to an embodiment of the present invention, FIG. 5B is a diagram showing a rhinitis prescription map according to the rhinitis protocol of an embodiment of the present invention, and FIG. 5A and FIG. 5D are diagrams showing medical questionnaires according to the rhinitis protocol of an embodiment of the present invention.

Referring to FIG. 5A, based on the patient's feedback information about medical inquiry content associated with the rhinitis protocol disclosed in FIG. 5C and FIG. 5D below, the rhinitis protocol may classify the patient into watery, mucous, purulent, and dry types, and determine guiding herbs and combination herbs associated with the guiding herbs according to aggravating and alleviating factors.

Additionally, as clinical symptoms of rhinitis, it may be classified into allergic and hypertrophic (for example, sinusitis) types. Clear and thin rhinorrhea with sneezing, white and sticky rhinorrhea, and yellow and thick rhinorrhea may be considered as allergic rhinitis symptoms, being symptoms of watery, mucous, and purulent rhinitis respectively. As symptoms of dry rhinitis, there may be no rhinorrhea, the inside of the nose may be very dry, or occasionally there may be nasal sores or dry crusts visible inside the nose.

Specifically, in cases where rhinitis is aggravated by cold stimuli, relieved by heat stimuli, and typically seasonal, at least one of ‘Ephedra’ and ‘Asarum’ may be used as primary guiding herbs, with ‘Ginger’ or ‘Cistanche’ as secondary guding herbs. Furthermore, in this rhinitis protocol, when the condition is mucous in nature, aggravated by cold stimuli, relieved by heat stimuli, and typically seasonal, ‘Pinellia’ is used as the primary guiding herb, with ‘Aurantii fructus Immaturus’ as the secondary guiding herb. In cases where it is purulent in nature, largely unaffected by hot or cold stimuli, and perennial, ‘Platycodon’ is used as the primary guiding herb, with ‘Coicis Semen’ as the secondary guiding herb. In cases where it is dry in nature, triggered and aggravated by dry environments, and relieved in humid environments, ‘Scutellaria’ is used as the primary guiding herb, with ‘Ophiopogon’, ‘Schisandra’, and ‘Armeniacae Semen’ as secondary guiding herbs.

Additionally, in the aforementioned serous rhinitis, at least one of ‘Pinellia’, ‘Schisandra’, and ‘Ginger’ is used as a combination herb, and in cases where nasal congestion is present, at least one of Magnolia Flower (Xin Yi), Ligusticum (Chuan Xiong), Xanthium (Cang Er Zi), and Angelica Dahurica (Bai Zhi) is used as a combination herb, and in cases of purulent or hypertrophic rhinitis, at least one of ‘Gypsum’, ‘Scutellaria’, ‘Pueraria’, and ‘Snake Gourd’ may be used as a complementary heat-clearing herb.

The aforementioned rhinitis protocol may be approached comprehensively, incorporating not only the Shanghan Jinkui prescriptions but also later-generation prescriptions, and rhinitis may be broadly categorized into serous and purulent types. When the nasal discharge is clear and watery, it may be classified as serous, and when the nasal discharge is yellow and viscous, it may be classified as purulent.

Dry type refers to a condition with no nasal discharge and a dry state. Dry rhinitis is often treated as a blind spot of Shanghan Jinkui prescriptions, and is frequently treated with later-generation prescriptions. Nasal congestion may appear in all types: serous, mucous, purulent, and dry.

In actual clinical practice, it is often not clearly observable whether the nasal discharge is serous or purulent; however, in such cases, distinction may be made by identifying the aggravating and relieving factors of serous and purulent rhinitis. In particular, symptoms of serous rhinitis may be triggered and aggravated by cold stimuli, and may be relieved and alleviated by heat stimuli. When exposed to cold stimuli, runny nose, sneezing, or nasal congestion may be triggered and aggravated. In contrast, purulent rhinitis is not significantly affected by hot or cold stimuli. Therefore, serous rhinitis is often triggered and aggravated during seasonal changes and cold seasons. Purulent rhinitis often does not significantly worsen during seasonal changes or cold seasons and frequently persists even in summer, making it commonly perennial in nature. Through the questionnaires shown in FIG. 5c and FIG. 5d below, serous and purulent rhinitis may be distinguished easily, simply, and clearly.

As mentioned earlier, if it is serous rhinitis, ‘Ephedra’ and ‘Asarum’ may be used as primary guiding herbs. Preferably, if conditions allow for the use of ‘Ephedra’, it may be considered over ‘Asarum’. However, if conditions contraindicate the use of ‘Ephedra’, ‘Asarum’ may be prescribed instead. Additionally, prescriptions for treating serous rhinitis may combine ‘Ephedra’ or ‘Asarum’ with ‘Pinellia’, ‘Schisandra’, and ‘Ginger’. Referring to FIG. 5b, if it is serous rhinitis, prescriptions such as Small Green Dragon Decoction types, Snake Gourd and Ephedra Decoction, and Scallion and Ginger with Asarum and Pinellia Decoction types may be prescribed. Meanwhile, if it is purulent rhinitis, ‘Platycodon’ may be used as the primary guiding herb. As there are cases where the nature of nasal discharge spans both purulent and serous characteristics, this is referred to as mucous rhinitis.

If it is mucous rhinitis, ‘Aurantii Fructus Immaturus’ and ‘Pinellia’ may be used as primary guiding herbs. ‘Pinellia’ may be combined in prescriptions treating serous rhinitis. ‘Aurantii Fructus Immaturus’ may be combined alone in prescriptions treating mucous rhinitis. It may be used in mucous rhinitis prescriptions when combined in Cinnamon Twig Decoction minus Peony plus Aurantii Fructus Immaturus.

If it is dry rhinitis, ‘Scutellaria’ may be used as the primary guiding herb. Additionally, in rhinitis treatment prescriptions, heat-clearing herbs and nourishing herbs may be combined besides primary and secondary guiding herbs. Heat-clearing herbs such as Gypsum, Scutellaria, Pueraria, and Snake Gourd may be combined, and nourishing herbs such as Armeniacae Semen, Schisandra, and Ophiopogon may be combined. When heat-clearing herbs are combined, even in cases of serous rhinitis, symptoms of purulent and hypertrophic rhinitis may appear concurrently. When the main symptom of rhinitis is nasal congestion and the main prescription alone is insufficient, Magnolia Flower (Xin Yi), Ligusticum (Chuan Xiong), Xanthium (Cang Er Zi), and Angelica Dahurica (Bai Zhi) may be added. Preferably, ‘Magnolia Flower’ may be added.

Referring to FIG. 5B, rhinitis prescriptions may be determined by considering serous/mucous/purulent/dry types along with excess heat/between excess and deficiency/deficiency cold conditions. Excess heat (Shi Re) refers to symptoms where pathogenic qi is excessive and substantial, causing heat symptoms, deficiency cold (Xu Han) refers to symptoms where upright qi is deficient and internal cold symptoms appear concurrently, and between excess and deficiency refers to a state between excess heat and deficiency cold.

Referring to FIG. 5C, without limitation, the rhinitis questionnaire includes content asking about rhinitis symptoms, nasal discharge conditions, content related to aggravating and relieving factors of rhinitis, presence of nasal congestion, and other related content. The rhinitis questionnaire may be updated and modified through the aforementioned artificial intelligence learning.

FIG. 6A is a drawing showing an example of a dyspnea protocol according to an embodiment of the present invention, and FIG. 6B is a drawing showing a dyspnea prescription map according to the dyspnea protocol of an embodiment of the present invention.

Referring to FIGS. 6A and 6B, based on patient feedback information regarding the diagnostic questionnaire content related to dyspnea or asthma protocol, the dyspnea or asthma protocol may classify the patient into serous, purulent, mucous, or dry types, and determine guiding herbs according to aggravating and relieving factors.

Specifically, if it is serous type and conditions allow for the use of Ephedra, ‘Ephedra’ may be used as the primary guiding herb, and any one of Munhap-tang (Korean herbal decoction combining harmonizing herbs), Gamcho-mawhang-tang (Licorice-Ephedra Decoction), Sagan-mawhang-tang (Belamcanda and Ephedra Decoction), Bangki-hwangki-gamawhang-tang (Sinomenium, Astragalus and Ephedra Decoction), and Oedaesinbi-tang (Major Decoction for Body Revitalization) may be prescribed. Among these, Gamcho-mawhang-tang and Munhap-tang may be frequently prescribed, and Mahwang-buja-gamcho-tang, Sagan-mahwang-tang, Moryo-tang, Bangki-hwangki-gamawhang-tang, Hubak-mahwang-tang, and Oedaesinbi-tang may be occasionally prescribed. If it is serous type but conditions contraindicate the use of Ephedra, ‘Cistanche’ may be used as the primary guiding herb and ‘Jujube’ as the secondary guiding herb, and any one of Jeong-ryeok-daejosapye-tang (Cistanche and Jujube Four-Lung Decoction), Gichoryok-hwang-hwan (Energy-Promoting Cistanche Yellow Pill), or Daeham-hyoong-hwan (Major Chest-Containing Pill) containing Cistanche may be prescribed.

Jeong-ryeok-daejosapye-tang may be frequently prescribed, while Gichoryok-hwang-hwan and Daeham-hyoong-hwan may be occasionally prescribed. In cases where it is purulent type and not significantly affected by hot or cold stimuli, ‘Platycodon’ may be used as the guiding herb, and Bae-nong-tang (Pus-Expelling Decoction) may be prescribed.

In cases where it is mucous type with chest discomfort, at least one of ‘Trichosanthes Seed’, ‘Magnolia Bark’, or ‘Apricot Kernel’ may be used as primary guiding herbs, with ‘Pinellia’ or ‘Apricot Kernel’ as secondary guiding herbs, and any one of Soham-hyoong-tang (Minor Chest-Containing Decoction), Gwaru-haebaek-banha-tang (Trichosanthes, Chinese Chive, and Pinellia Decoction), Banhahoobak-tang (Pinellia and Magnolia Bark Decoction), Sojadodam-gangki-tang (Minor Purple-Addressing Phlegm and Descending Qi Decoction), or Gyejigahoo-bakhaengja-tang (Cinnamon Twig plus Magnolia Bark and Apricot Kernel Decoction) may be prescribed.

Among these, Banhahoobak-tang (Pinellia and Magnolia Bark Decoction) and Gyejigahoo-bakhaengja-tang (Cinnamon Twig plus Magnolia Bark and Apricot Kernel Decoction) may be frequently prescribed, while Soham-hyoong-tang (Minor Chest-Containing Decoction), Gwaru-haebaek-banha-tang (Trichosanthes, Chinese Chive, and Pinellia Decoction), Sojadodam-gangki-tang (Minor Purple-Addressing Phlegm and Descending Qi Decoction) and Oedaesinbi-tang (Major Decoction for Body Revitalization) may be occasionally prescribed.

Among these, Banhahoobak-tang (Pinellia and Magnolia Bark Decoction) and Gyejigahoo-bakhaengja-tang (Cinnamon Twig plus Magnolia Bark and Apricot Kernel Decoction) may be frequently prescribed, while Soham-hyoong-tang (Minor Chest-Containing Decoction), Gwaru-haebaek-banha-tang (Trichosanthes, Chinese Chive, and Pinellia Decoction), Sojadodam-gangki-tang (Minor Purple-Addressing Phlegm and Descending Qi Decoction), and Oedaesinbi-tang (Major Decoction for Body Revitalization) may be occasionally prescribed.

In cases where it is dry type with chest discomfort and chest stagnation, ‘Citrus Peel’ may be used as the guiding herb, and in cases where it is dry type with neck and lumbar edema, ‘Stephania’ may be used as the guiding herb, and any one of Citrus Peel and Bamboo Leaf Decoction), Citrus Peel, Immature Citrus, and Fresh Ginger Decoction, Citrus Peel Decoction), Citrus Peel, Rhubarb, and Mint Decoction, Wood Stephania Decoction, Wood Stephania minus Gypsum plus Poppy Decoction, Energy-Promoting Cistanche Yellow Pill), or Stephania and Astragalus plus Ephedra Decoction) may be prescribed.

Among these, Citrus Peel and Bamboo Leaf Decoction and Wood Stephania Decoction may be frequently prescribed, while Citrus Peel, Immature Citrus, and Fresh Ginger Decoction, Citrus Peel Decoction, Citrus Peel, Rhubarb, and Mint Decoction, Wood Stephania minus Gypsum plus Poppy Decoction, Energy-Promoting Cistanche Yellow Pill, and Stephania and Astragalus plus Ephedra Decoction) may be occasionally prescribed.

FIG. 7A shows an example of a chronic cough protocol according to an embodiment of the present invention, FIG. 7b shows a chronic cough prescription map according to the chronic cough protocol of an embodiment of the present invention, and FIGS. 7C and 7D shows diagnostic questionnaire content according to the chronic cough protocol of an embodiment of the present invention.

Referring to FIG. 7A, the chronic cough protocol has similarities with the rhinitis protocol, and based on patient feedback information regarding the diagnostic questionnaire content related to the chronic cough protocol disclosed in FIGS. 7C and 7D below, the chronic cough protocol may classify the patient into serous, mucous, purulent, and dry types, and determine guiding herbs and their associated combination herbs according to aggravating and relieving factors.

Specifically, in cases where it is serous type with cold stimulus triggering aggravation and heat stimulus causing relief, at least one of ‘Ephedra’ or ‘Asarum’ may be used as primary guiding herbs, and at least one of ‘Ginger’ and ‘Cistanche’ may be used as secondary guiding herbs, and in cases where it is mucous type with cold stimulus triggering aggravation and heat stimulus causing relief, ‘Pinellia’ may be used as the primary guiding herb, and at least one of ‘Aurantii Fructus Immaturus’, ‘Inula’ and ‘White Mustard Seed’ may be used as secondary guiding herbs, and in cases where it is purulent type and largely unaffected by hot or cold stimuli, ‘Platycodon’ may be used as the primary guiding herb, and at least one of ‘Trichosanthes Seed’, ‘Fritillaria’, ‘Angelica’ and ‘Bamboo Leaf’ may be used as secondary guiding herbs, and in cases where it is dry type, triggered and aggravated by dry environments and relieved in humid environments, ‘Scutellaria’ may be used as the primary guiding herb, and at least one of ‘Schisandra’, ‘Apricot Kernel’ and ‘Prepared Rehmannia’ may be used as secondary guiding herbs. In the chronic cough protocol, at least one of ‘Perilla Leaf’, ‘Purple Perilla’, ‘Self Heal’, ‘Coltsfoot’, ‘Mulberry Leaf’, and ‘Mulberry Root Bark’ may be prescribed as complementary antitussive and expectorant herbs.

Referring to FIG. 7B, in cases where it is serous type and Ephedra is used as the primary guiding herb, for patients with heat-type constitution, Wolbi-gabanha-tang (Modified Yuebi Decoction with Pinellia), Mahaeng-gamseok-tang (Ephedra, Apricot Kernel, Licorice, and Gypsum Decoction), and Sagan-mahwang-tang (Snake Gourd and Ephedra Decoction) may be prescribed, and for patients with cold-type constitution, Socheongryong-tang-ryu (Minor Blue Dragon Decoction-type) and Mahwang-buja-sesin-tang (Ephedra, Aconite, and Asarum Decoction) may be prescribed. Additionally, in cases where it is serous type and Asarum is used as the primary guiding herb, Younggam-gangmi-sinha-tangryu (Scallion and Ginger with Asarum and Pinellia Decoction-type) may be prescribed, and in cases where it is serous type and Cistanche is used as the primary guiding herb, Jeongryeok-daejosa-pye-tang (Cistanche and Jujube Four-Lung Decoction) may be prescribed.

Additionally, in cases where it is mucous type and ‘Pinellia’ is used as the primary guiding herb, Soham-hyoong-tang (Minor Chest-Containing Decoction), Haengso-tang (Apricot Kernel and Minor Decoction), Soja-dodam-ganggi-tang (Minor Purple-Addressing Phlegm and Descending Qi Decoction), Samso-eum (Three Minor Decoction), and Ginseng Toxin-Defeating Powder may be prescribed. In cases where it is purulent type and ‘Platycodon’ is used as the primary guiding herb, one of Mulberry and Chrysanthemum Decoction, Pus-Expelling Powder, Pus-Expelling Decoction, or Urgent Pus-Expelling Powder Decoction may be prescribed. In cases where it is dry type and one of ‘Ophiopogon’, ‘Schisandra’, ‘Apricot Kernel’, or ‘Prepared Rehmannia’ is used as the primary guiding herb, one of Clear Dryness and Rescue the Lung Decoction, Bamboo Leaf and Gypsum Decoction, Ophiopogon Decoction, Bupleurum plus Honey Decoction, Four Stations plus Honey Powder, Jinmu Plus Sweet Herb Decoction, Poria and Cinnamon with Honey Decoction, Cinnamon Twig Decoction with Magnolia Bark and Apricot Kernel or Rehmannia Pill for Pulse Stabilization may be prescribed.

Referring to FIGS. 7C and 7D, the diagnostic questionnaire for the chronic cough protocol may include questions about the presence of nasal discharge, sputum, and cough, as well as questions related to the condition of nasal discharge and sputum. Without limitation, the inquiry content shown in the diagnostic questionnaire for the chronic cough protocol may be updated and modified through the aforementioned artificial intelligence learning.

FIGS. 8A-8D are drawings showing examples of a musculoskeletal pain disease protocol according to an embodiment of the present invention, and FIGS. 8E-8G are drawings showing musculoskeletal pain disease prescriptions according to the musculoskeletal pain disease protocol of an embodiment of the present invention.

Referring to FIGS. 8A-8D, based on patient feedback information regarding the diagnostic questionnaire content related to the disclosed musculoskeletal pain disease protocol, the musculoskeletal pain disease protocol classifies the patient into primary and secondary pathogenic factors, where the primary pathogenic factors are categorized into decreased body temperature, increased body temperature, edema, and muscle tension. The secondary pathogenic factors are categorized into blood stasis, nutritional deficiency, phlegm-dampness, qi stagnation, and spirit, where the primary pathogenic factors' guiding herbs are determined according to decreased body temperature, increased body temperature, edema, and muscle tension, and the secondary pathogenic factors' guiding herbs are determined according to blood stasis, nutritional deficiency, phlegm-dampness, qi stagnation, and spirit.

Specifically, in cases where it is a primary pathogenic factor and decreased body temperature, ‘Cinnamon Twig’ is used as the primary guiding herb, and ‘Evodia’ or ‘Aconite’ is used as the secondary guiding herb, and in cases where it is a primary pathogenic factor and increased body temperature, at least one of ‘Gypsum’, ‘Sweet Sedge’, ‘Scutellaria’ is used as the primary guiding herb, and ‘Bupleurum’, ‘Coptis’ or ‘Phellodendron’, ‘Gardenia’, or ‘Anemarrhena’ is used as the secondary guiding herb, and in cases where it is a primary pathogenic factor and edema, at least one of ‘Atractylodes’, ‘Stephania’, ‘Sweet Sedge’ is used as the primary guiding herb, and ‘Astragalus’ or ‘Ephedra’ is used as the secondary guiding herb, and in cases where it is a primary pathogenic factor and muscle tension, ‘Jujube’ or ‘Peony’ may be used as the guiding herb.

Meanwhile, in cases where it is a secondary pathogenic factor with blood stasis and deficiency pattern, ‘Peony’ is used as the guiding herb, and in cases where it is a secondary pathogenic factor with blood stasis and excess pattern, ‘Rhubarb’ is used as the guiding herb, and in cases where it is a secondary pathogenic factor and nutritional deficiency, ‘Rehmannia’ is used as the guiding herb, and in cases where it is a secondary pathogenic factor and phlegm-dampness, at least one of ‘Pinellia’, ‘Citrus Peel’, ‘Arisaema’ is used as the guiding herb, and in cases where it is a secondary pathogenic factor and qi stagnation, at least one of ‘Cyperus’, ‘Perilla Leaf’, ‘Citrus Peel’, ‘Lindera’, ‘Citrus unshiu Peel’ is used as the guiding herb, and in cases where it is a secondary pathogenic factor and spirit with excitation, ‘Coptis’ is used as the primary guiding herb, with ‘Oyster Shell’ as the secondary guiding herb, and Baihu Tang-type formulas are used, and in cases where it is a secondary pathogenic factor and spirit with depression, at least one of ‘Poria’ or ‘Costus+Gardenia’ is used as the primary guiding herb, and ‘Dragon's Bone’ may be used as the secondary guiding herb.

Referring to FIGS. 8E-8G, prescriptions based on the disclosed musculoskeletal pain disease protocol may be categorized into frequently used prescriptions, moderately used prescriptions, and less frequently used prescriptions, and may be prescribed according to heat constitution, cold constitution, and between cold and heat constitution.

Referring to FIG. 8E, based on frequently used prescriptions, for patients with cold constitution without edema, any one of Yellow Bell Decoction, Poria and Cinnamon with Licorice Decoction, Cinnamon Twig Decoction, Evodia Decoction, Bupleurum minus Ginseng plus Cinnamon Twig Decoction, Bupleurum and Cinnamon Twig Decoction, Poria, Cinnamon, Atractylodes, and Licorice Decoction may be prescribed, and for patients with constitution between cold and heat without edema, any one of Pinellia Heart-Draining Decoction, Fresh Ginger Heart-Draining Decoction, Licorice and Four minus Ginseng Decoction, Minor Bupleurum Decoction, Minor Chest Relief Decoction may be prescribed, and for patients with heat constitution without edema, any one of Ten Jujube Decoction, Peony and Licorice Decoction, Ephedra and Coix Decoction may be prescribed.

For patients with cold constitution and slight edema, any one of Coptis Decoction, Cinnamon Twig Decoction with Pueraria and Pinellia, Ginseng, Atractylodes, Poria, and Glycyrrhiza Decoction, Ephedra Decoction may be prescribed, and for patients with constitution between cold and heat and slight edema, Minor Chest-Relieving Decoction is prescribed, and for patients with heat constitution and slight edema, any one of Kansui and Pinellia Decoction, Yuebi Decoction, Modified Yuebi Decoction with Pinellia, Wind-Dispelling and Blood-Activating Decoction may be prescribed.

For a patient with slight edema and a cold constitution, Minor Chest-Relieving Decoction is prescribed. For a patient with slight edema and a warm constitution, Kansui and Pinellia Decoction, Yuebi Decoction, Modified Yuebi Decoction with Pinellia, or Wind-Dispelling and Blood-Activating Decoction may be prescribed. For a patient with significant edema and a cold constitution, Fangji Huangqi Decoction with Cinnamon Twig, Ephedra and Atractylodes Decoction, or Fangji Fuling Decoction may be prescribed. For a patient with significant edema and a slightly cold constitution, Fangji Huangqi Decoction or Modified Gunggui Zoyueum may be prescribed. For a patient with significant edema and a warm constitution, Kansui and Pinellia Decoction, Modified Yuebi Decoction with Atractylodes, or Wind-Dispelling and Blood-Activating Decoction may be prescribed.

Referring to FIG. 8F, for a patient with musculoskeletal pain based on traditional moderate prescriptions and without edema and with a cold constitution, any of the following prescriptions may be prescribed: Yukmuhwanggeum Decoction, Cinnamon Twig Decoction with Additional Cinnamon Twig, Cinnamon Twig and Aconite Decoction, Shin Decoction, Aconite and Cinnamon Twig Decoction, Cinnamon Twig and Poria Pill, Cinnamon Twig and Peony Decoction, Cinnamon Twig and Rhubarb Decoction, Minor Construct the Middle Decoction, Cinnamon Twig without Peony Decoction, Daechungnyong Decoction, Houpo Seven-Substance Decoction, Duhuo and Parasite Decoction, Great Wind-Proofing Decoction, Modified Great Tonifying Decoction, Aconite and Coptis Decoction, Palmi Pill, White Tiger with Cinnamon Twig Decoction, or Taoren Chengqi Decoction. For a patient with musculoskeletal pain, without edema, and a slightly cold constitution, any of the following prescriptions may be prescribed: Galgeun Decoction, Galgeun with Pinellia Decoction, Ssanghwa Decoction, Taekran Decoction, Haenggi Hyangso Powder, Oyaksunki Powder, Hoesoo Powder, Samhap Decoction, Palmul Decoction, Yeoshin Decoction, Iban Powder, or Sikyung with Pinellia Decoction. For a patient with musculoskeletal pain, without edema, and a warm constitution, any of the following prescriptions may be prescribed: Daesihotang, Scutellaria Decoction with Pinellia and Fresh Ginger, Galgeunlian Decoction, Baekho Decoction, Baekho with Ginseng Decoction, Yukmi with Gardenia, or Yeongseonjeitong Drink.

For a patient with slight edema and a cold constitution, any of the following prescriptions may be prescribed: Cinnamon Twig with Astragalus Decoction, Astragalus and Cinnamon Twig Five-Substance Decoction, Cinnamon Twig with Magnolia Bark and Apricot Seed Decoction, Cinnamon with Ginger, Jujube, Scutellaria, Fresh Ginseng, and Aconite Decoction, Cinnamon Twig Two and Ephedra One Decoction, Cinnamon Twig Two and Yuebi One Decoction, Aconite and Coptis Decoction, Rhubarb, Aconite, and Asarum Decoction, Ginger, Atractylodes, and Licorice Decoction, or Palmi Pill.

For a patient with slight edema and a slightly cold constitution, either Cinnamon Twig Decoction without Cinnamon and with Atractylodes and Poria or Ginger, Atractylodes, and Licorice Decoction may be prescribed. For a patient with slight edema and a warm constitution, any of the following prescriptions may be prescribed: Major Chest-Relieving Decoction, Major Chest-Relieving Pill, or Rhubarb and Kansui Decoction.

For a patient with significant edema and a cold constitution, any of the following prescriptions may be prescribed: Cinnamon Twig with Astragalus Decoction, Astragalus and Cinnamon Twig Five-Substance Decoction, Danggui Sayeok Decoction with Evodia and Fresh Ginger, or Fangji Astragalus Decoction with Peony. For a patient with significant edema and a slightly cold constitution, Fangji Astragalus Decoction with Ephedra or Binso Powder may be prescribed. For a patient with significant edema and a warm constitution, any of the following prescriptions may be prescribed: Major Chest-Relieving Decoction, Major Chest-Relieving Pill, or Rhubarb and Kansui Decoction.

Referring to FIG. 8G, for a patient with musculoskeletal pain based on traditional moderate prescriptions and without edema and with a cold constitution, any of the following prescriptions may be prescribed: Gueok Decoction, Jinmu Decoction, Aconite Decoction, Chaihu with Dragon Bone and Oyster Decoction, Chaihu Cinnamon Ginger Decoction, Tokuwa Root Powder, Gualou Cinnamon Decoction, Cinnamon Twig with Kudzu Decoction, Astragalus Construct the Middle Decoction, Peony Licorice Aconite Decoction, Zhi Licorice Decoction, Cinnamon Twig without Peony with Aconite Decoction, Cinnamon Twig Aconite Decoction, Licorice Aconite Decoction, Atractylodes Aconite Decoction, Minor Bluegreen Dragon Decoction, Samgi Drink, Ginger Aconite Decoction, Cinnamon Ginger Bitter Orange Decoction, Ojeok Powder, Deer Antler Great Tonifying Decoction, Daeyeongjeon, or Samgi Drink.

For a patient with musculoskeletal pain, without edema and with a slightly cold constitution, any of the following prescriptions may be prescribed: Coptis and Donkey Hide Gelatin Decoction, Inula and Haizaitang Decoction, Ginger Licorice Decoction, Tangerine Peel and Bamboo Shavings Decoction, Chaihu with Magnesium Sulfate Decoction, Chaihu without Gold with Peony Decoction, Chaihu without Gold with Poria Decoction, Chaihu without Pinellia with Ginseng and Kudzu Decoction, Chaihu without Pinellia with Gualou Fruit Decoction, Chaihu with Dragon Bone and Oyster Decoction, Mai Men Dong Decoction, Angelica and Mugwort Decoction, Licorice and Ginger Decoction, Polyporus Decoction, Pinellia and Ginseng Decoction, Major Construct the Middle Decoction, Regulate the Stomach and Purge Decoction, or Sokmyeong Decoction. For a patient with musculoskeletal pain, without edema and with a warm constitution, any of the following prescriptions may be prescribed: Bitter Orange and Peony Powder, Sayeok Powder, Gardenia and Phellodendron Decoction, Gardenia and Ginger Decoction, Rhubarb and Moutan Bark Decoction, Pulsatilla Decoction with Donkey Hide Gelatin and Licorice, Three-Substance Scutellaria Decoction, or Preserve Yin Decoction.

For a patient with slight edema and a cold constitution, any of the following prescriptions may be prescribed: Cinnamon Twig Decoction without Peony and with Atractylodes, Danggui Sayeok Decoction, Cinnamon Twig and Anemarrhena Decoction, Aconite Decoction, Three-Yellow Decoction, or Coix Seed Decoction. For a patient with slight edema and a slightly cold constitution, any of the following prescriptions may be prescribed: Descurainia and Jujube to Drain the Lungs Decoction, Seoye Pill, Shiling Decoction, or Ephedra, Apricot Kernel, Coix, and Licorice Decoction. For a patient with slight edema and a warm constitution, Munhap Decoction may be prescribed.

For a patient with significant edema and a cold constitution, any of the following prescriptions may be prescribed: Aconite Decoction, Danggui and Peony Powder, or Poria and Alisma Decoction. For a patient with significant edema and a slightly cold constitution, any of the following prescriptions may be prescribed: Shiling Decoction, Muli and Alisma Powder, or Daeganghwal Decoction. For a patient with significant edema and a warm constitution, Qingre Shishi Decoction may be prescribed.

As described above, a personalized oriental medicine diagnosis and prescription device may be provided by extracting herbal ingredients based on the patient's responses that match the disease protocol and consultation content. By deriving a candidate pool of herbal prescriptions that align with the extracted ingredients, it is possible to automatically offer candidate prescriptions tailored to the symptoms of each patient. Additionally, standardized and quantified prescription guidelines may be provided to the patient or practitioner.

In this specification, preferred embodiments of the present invention have been disclosed, and although specific terms have been used, they are employed solely in a general sense to facilitate explanation and understanding of the invention's technical content. They are not intended to limit the scope of the invention. It is evident to those skilled in the art that, in addition to the embodiments disclosed herein, other variations based on the technical spirit of the present invention may also be implemented. Those skilled in the art will understand that the traditional medicine diagnosis and prescription device and method described with reference to FIGS. 1-8F may be variously substituted, modified, and altered without departing from the technical spirit of the present invention.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. An apparatus for an oriental medicine diagnosis and prescription, comprising:
an identification unit that receives patient login information from a user terminal and identifies the patient based on the received login information;
a first medical inquiry execution unit that requests information on the illness or condition the identified patient wishes to treat from the user terminal;
an inquiry selection unit that, based on the patient's response to the request, selects one among multiple medical inquiry contents that match with a plurality of disease protocols;
a second medical inquiry execution unit that transmits the selected inquiry content to the user terminal in a predetermined step-by-step sequence;
a prescription candidate group derivation unit that extracts herbal ingredients by considering the disease protocol matching the patient's response to the request and the patient's response corresponding to the selected inquiry content, and derives a candidate pool of herbal prescriptions matching the extracted ingredients; and
a prescription provision unit that lists the prescription candidate pool and provides it to the user terminal or to the terminal of the corresponding practitioner.

2. The apparatus of claim 1,
wherein the disease protocol includes at least one of insomnia protocol, psychiatry protocol, allergic or non-allergic rhinitis protocol, cold protocol, dyspnea or asthma protocol, chronic cough protocol, facial nerve paralysis protocol, cerebrovascular disease aftereffects protocol, dysmenorrhea protocol, gynecology protocol, lumbar disc herniation (lower back disc) protocol, other musculoskeletal pain protocol, functional dyspepsia protocol, or other digestive system protocols,
wherein the psychiatry protocol includes at least one of panic disorder, anxiety disorder, obsessive-compulsive disorder, depression, schizophrenia, tic disorder, or epilepsy,
wherein the gynecology protocol includes at least one of infertility, amenorrhea, or abnormal uterine bleeding,
wherein the other musculoskeletal pain protocol includes at least one of the following: cervical disc herniation, spinal stenosis, arthritis, or frozen shoulder,
wherein the other digestive system protocol includes at least one of constipation, diarrhea, or gastritis.

3. The apparatus of claim 2,
wherein the insomnia protocol, based on the selected medical inquiry content, categorizes the patient into an inducing type or aggravating type, and determines a guiding herb and weak emotion according to the duration and frequency of the patient's symptoms,
in case of an inducing type with a long (chronic), severe, and frequent occurrence of symptoms, the guiding herb used is “Coptis” (Huanglian), and the weak emotion is characterized by states of agitation and anger, and
in case of an inducing type with a short (acute), mild, and occasional occurrence of symptoms, at least one of the guiding herbs “Oyster Shell” (Muli) or “Dragon Bone” (Longgu) is used, and the weak emotion is characterized by states of anxiety or fear,
in case of an aggravating type with a long (chronic), severe, and frequent occurrence of symptoms, the guiding herb used is either “Citrus Seed” (Xiangshi) or “Gardenia” (Zhizi), and the weak emotion is characterized by states of resentment and sadness,
in case of an aggravating type with a short (acute), mild, and occasional occurrence of symptoms, the guiding herb used is either “Poria” (Fuling) or “Ziziphus Seed” (Suanzaoren), and the weak emotion is characterized by a state of worry.

4. The apparatus of claim 3, wherein the “Coptis” (Huanglian) and “Oyster Shell” (Muli) are prescribed for patients with a yin constitution, and the “Citrus Seed” (Xiangshi), “Gardenia” (Zhizi), “Poria” (Fuling), “Ziziphus Seed” (Suanzaoren), and “Dragon Bone” (Longgu) are prescribed for patients with a yang constitution.

5. The apparatus of claim 4,
wherein the rhinitis protocol, based on the selected medical inquiry content, categorizes the patient into serous, mucoid, purulent, or dry types and determines a guiding herb and associated formulation herbs according to aggravating and relieving factors,
in case of the serous type with cold stimulation as an aggravating factor, at least one of the primary guiding herbs “Ephedra” (Mahuang) or “Asarum” (Xixin) is used, along with “Dried Ginger” (Ganjiang) as a secondary guiding herb,
in case of the serous type with warm stimulation as a relieving factor, at least one of the primary guiding herbs “Ephedra” (Mahuang) or “Asarum” (Xixin) is used, and “Descurainia Seed” (Tinglizi) is used as a secondary guiding herb,
in case of the mucoid type with seasonal characteristics, “Pinellia” (Banxia) is used as the primary guiding herb, and “Gleditsia Fruit” (Zaojia) is used as the secondary guiding herb,
in case of the purulent type with minimal sensitivity to cold or warm stimuli and year-round symptoms, “Platycodon” (Jiegeng) is used as the primary guiding herb, and “Coix Seed” (Yiyiren) is used as the secondary guiding herb,
in case of the dry type that is aggravated in a dry environment and relieved in a humid environment, “Scutellaria” (Huangqin) is used as the primary guiding herb, and “Ophiopogon” (Maimendong), “Schisandra” (Wuweizi), and “Apricot Seed” (Xingren) are used as secondary guiding herbs.

6. The apparatus of claim 5,
wherein at least one of “Pinellia” (Banxia), “Schisandra” (Wuweizi), or “Dried Ginger” (Ganjiang) is used as a formulation herb in the serous rhinitis type,
if nasal congestion is present in the serous rhinitis type, at least one of “Magnolia Flower” (Xinyi), “Ligusticum” (Chuanxiong), “Xanthium” (Cang′erzi), or “Angelica Dahurica” (Baizhi) is used as a formulation herb,
in case of the purulent rhinitis type, at least one of the heat-clearing formulation herbs “Gypsum” (Shigao), “Scutellaria” (Huangqin), “Pueraria” (Gegen), or “Belamcanda” (Shegan) is used.

7. The apparatus of claim 2,
wherein the dyspnea or asthma protocol, based on the selected medical inquiry content, categorizes the patient into serous, purulent, mucoid, or dry types and determines a guiding herb according to aggravating and relieving factors. For the serous type, if conditions permit the use of Ephedra, “Ephedra” (Mahuang) is used as the primary guiding herb,
if the serous type does not permit the use of Ephedra, “Descurainia Seed” (Tinglizi) is used as the primary guiding herb, with “Jujube” (Dazao) as a secondary guiding herb. For the purulent type with minimal sensitivity to cold or warm stimuli, “Platycodon” (Jiegeng) is used as the guiding herb,
in case of the mucoid type with chest discomfort, at least one of “Trichosanthes Fruit” (Gualou Shi), “Magnolia Bark” (Houpo), or “Apricot Seed” (Xingren) is used as the primary guiding herb, and “Pinellia” (Banxia) or “Apricot Seed” (Xingren) is used as the secondary guiding herb,
in case of the dry type with chest discomfort and intercostal stagnation, “Tangerine Peel” (Jupi) is used as the guiding herb. For the dry type with pretibial pitting edema, “Stephania” (Fangji) is used as the guiding herb.

8. The apparatus of claim 7,
wherein in case of the serous type of dyspnea or asthma where “Ephedra” (Mahuang) is used as the guiding herb, one of the following may be prescribed: Munhap Decoction, Licorice and Ephedra Decoction, Ephedra, Aconite, and Licorice Decoction, Belamcanda and Ephedra Decoction, Fangji and Astragalus with Ephedra Decoction, or Odaesinba Decoction,
in case of the serous type of dyspnea or asthma where “Descurainia Seed” (Tinglizi) is used as the guiding herb, one of the following may be prescribed: Descurainia and Jujube to Drain the Lungs Decoction, Gichoryeokhwang Pill, or Major Chest-Relieving Pill,
in case of the mucoid type of dyspnea or asthma where “Pinellia” (Banxia), “Trichosanthes Fruit” (Gualou Shi), “Magnolia Bark” (Houpo), or “Perilla Leaf” (Soyeop) is used as the guiding herb, one of the following may be prescribed: Minor Chest-Relieving Decoction, Trichosanthes Fruit, Chinese Chive, and Pinellia Decoction, Pinellia and Magnolia Bark Decoction, or Perilla Fruit to Dispel Phlegm and Descend Qi Decoction,
in case of the dry type of dyspnea or asthma where “Apricot Seed” (Xingren), “Tangerine Peel” (Jupi), or “Stephania” (Fangji) is used as the guiding herb, one of the following may be prescribed: Cinnamon Twig with Magnolia Bark and Apricot Seed Decoction, Poria, Apricot Seed, and Licorice Decoction, Tangerine Peel and Bamboo Shavings Decoction, Tangerine Peel, Bitter Orange, and Fresh Ginger Decoction, Stephania Decoction, Modified Stephania Decoction without Gypsum but with Licorice, Gichoryeokhwang Pill, or Fangji and Astragalus with Ephedra Decoction.

9. The apparatus of claim 2,
wherein the chronic cough protocol, based on the selected medical inquiry content, categorizes the patient into serous, mucoid, purulent, or dry types and determines a guiding herb and associated formulation herbs according to aggravating and relieving factors,
in case of the serous type with cold stimulation as an aggravating factor, at least one of the primary guiding herbs “Ephedra” (Mahuang) or “Asarum” (Xixin) is used, along with at least one of the secondary guiding herbs “Dried Ginger” (Ganjiang) or “Descurainia Seed” (Tinglizi),
in case of the mucoid type with warm stimulation as a relieving factor, “Pinellia” (Banxia) is used as the primary guiding herb, along with at least one of the secondary guiding herbs “Gleditsia Fruit” (Zaojia), “Inula Flower” (Xuanfuhua), or “White Mustard Seed” (Baijiezi),
in case of the purulent type with minimal sensitivity to cold or warm stimuli, “Platycodon” (Jiegeng) is used as the primary guiding herb, along with at least one of the secondary guiding herbs “Trichosanthes Fruit” (Gualou Shi), “Fritillaria” (Beimu), “Hogfennel Root” (Qianhu), or “Bamboo Shavings” (Zhuru),
in case of the dry type that is aggravated in a dry environment and relieved in a humid environment, “Scutellaria” (Huangqin) is used as the primary guiding herb, with at least one of the secondary guiding herbs “Schisandra” (Wuweizi), “Apricot Seed” (Xingren), or “Cooked Rehmannia Root” (Shudihuang).

10. The apparatus of claim 2, wherein the chronic cough protocol includes at least one of “Perilla Leaf” (Soyeop), “Perilla Seed” (Soja), “Purple Root” (Ziwan), “Coltsfoot Flower” (Guandonghua), “Mulberry Leaf” (Sangyeop), or “Mulberry Root Bark” (Sangbaipi) as formulation antitussive and expectorant agents.

11. The apparatus claim 2,
wherein the musculoskeletal pain protocol, based on the selected medical inquiry content, categorizes the patient into primary and secondary pathological factors, and the primary factors include decreased body temperature (cold), increased body temperature (heat), edema (water), and muscle tension (spasm), while the secondary factors include blood stasis (stasis), deficiency of nourishment (dryness), damp-phlegm (phlegm), qi stagnation (stagnation), and spirit (shen),
wherein the guiding herb for the primary pathological factor is determined based on decreased body temperature (cold), increased body temperature (heat), edema (water), or muscle tension (spasm), and the guiding herb for the secondary pathological factor is determined based on blood stasis (stasis), deficiency of nourishment (dryness), damp-phlegm (phlegm), qi stagnation (stagnation), or spirit (shen),
in case of the primary pathological factor of decreased body temperature (cold), “Cinnamon Twig” (Guizhi) is used as the primary guiding herb, with “Evodia” (Wuzhuyu) or “Aconite” (Fuzi) as secondary guiding herbs,
in case of the primary pathological factor of increased body temperature (heat), at least one of “Gypsum” (Shigao), “Kansui” (Gansui), or “Scutellaria” (Huangqin) is used as the primary guiding herb, with one of “Bupleurum” (Chaihu), “Coptis” (Huanglian), “Phellodendron” (Huangbai), “Gardenia” (Zhizi), or “Anemarrhena” (Zhimu) as the secondary guiding herb,
in case of the primary pathological factor of edema (water), at least one of “Atractylodes” (Cangzhu), “Stephania” (Fangji), or “Kansui” (Gansui) is used as the primary guiding herb, with either “Astragalus” (Huangqi) or “Ephedra” (Mahuang) as the secondary guiding herb,
in case of the primary pathological factor of muscle tension (spasm), “Jujube” (Dazao) or “Peony” (Shaoyao) is used as the guiding herb,
in case of the secondary pathological factor of blood stasis with deficiency, “Peony” (Shaoyao) is used as the guiding herb,
in case of blood stasis with excess, “Rhubarb” (Dahuang) is used as the guiding herb,
in case of the secondary pathological factor of deficiency of nourishment (dryness), “Rehmannia” (Dihuang) is used as the guiding herb,
in case of damp-phlegm (phlegm) as a secondary factor, at least one of the following guiding herbs is used: “Pinellia” (Banxia), “Tangerine Peel” (Chenpi), or “Arisaema” (Nanshing),
in case of the secondary pathological factor of qi stagnation (stagnation), at least one of the following guiding herbs is used: “Cyperus” (Xiangfu), “Perilla Leaf” (Soyeop), “Tangerine Peel” (Chenpi), “Lindera” (Wuyao), or “Bitter Orange” (Zhike),
in case of the secondary pathological factor related to spirit (shen) with excessive yang nature, “Coptis” (Huanglian) is used as the primary guiding herb, with “Oyster Shell” (Muli) and Baihu Tang-type formulas as secondary guiding herbs,
in case of the secondary pathological factor related to spirit (shen) with excessive yin nature, at least one of “Poria” (Fuling), “Citrus Seed” (Xiangshi), or “Gardenia” (Zhizi) is used as the primary guiding herb, with “Dragon Bone” (Longgu) as the secondary guiding herb.

12. The apparatus of claim 1, wherein the prescription candidate group derivation unit derives a primary prescription candidate pool of herbal preparations that correspond to the extracted herbal ingredients, maps supportive evidence (Bangjeung) to the primary prescription candidate pool, and then differentiates this primary pool by grade and rank to derive a secondary prescription candidate pool of herbal preparations, in an oriental medicine diagnosis and prescription apparatus.

13. The apparatus of claim 1,
wherein the oriental medicine diagnosis and prescription apparatus further comprises a traditional medicine database that stores and retains information on each of the multiple disease protocols and the multiple medical inquiry contents matching these disease protocols,
wherein the traditional medicine database further stores each herbal prescription provided to individual patients and feedbacks regarding the degree of symptom improvement received from these patients for use in artificial intelligence learning.

14. The apparatus of claim 13, wherein the disease protocols and medical inquiry contents are updated and modified based on artificial intelligence learning.

15. A method of an oriental medical diagnosis and prescription, comprising:
receiving login information of a patient from a user terminal and identifying the patient based on the patient's login information;
requesting information about the disease or condition that the identified patient wants to be treated for from the user terminal;
selecting one of multiple inquiry contents matching multiple disease protocols according to the patient's response to the request;
transmitting the selected inquiry content to the user terminal step by step according to a predetermined order;
extracting medicinal herbs considering the disease protocol matching the patient's response to the request and the patient's response to the selected inquiry content, and deriving a prescription candidate group of at least one oriental medicine preparation corresponding to the extracted medicinal herbs; and
listing the prescription candidate group and providing it to the user terminal or the corresponding Korean medicine doctor's terminal.
